(19) United States
(12) Patent Application Publication
(10) Pub. No.: US 2013/0060154 A1
(43) Pub. Date: Mar. 7, 2013

(54) ATRIAL FIBRILLATION DECISION APPARATUS, AND METHOD AND PROGRAM FOR DECIDING PRESENCE OF ATRIAL FIBRILLATION

(71) Applicant: Masanori MORITA, Fussa-shi (JP)

(72) Inventor: Masanori MORITA, Fussa-shi (JP)

(73) Assignee: SEIKO EPSON CORPORATION, Tokyo (JP)

(21) Appl. No.: 13/604,064

(22) Filed: Sep. 5, 2012

(30) Foreign Application Priority Data
Sep. 7, 2011 (JP) 2011-194904
Aug. 24, 2012 (JP) 2012-184978

(51) Int. Cl. A61B 5/046; A61B 5/0255; A61B 5/0456

(52) U.S. Cl. 600/501; 600/518

(57) ABSTRACT

An atrial fibrillation decision apparatus includes an acquisition unit that acquires a detected waveform signal indicating a detection result of an electrocardiogram or pulse wave, an RR interval calculation unit that calculates a parameter corresponding to the RR interval with respect to each frame on the basis of a spectrum of each frame obtained through frequency analysis of the detected waveform signal acquired, and an RR waveform signal that indicates a temporal change of the parameter, a power calculation unit that calculates a temporal change of power of a predetermined frequency band of the RR waveform signal, and a decision unit that decides whether the calculated power satisfies a specific condition and outputs information indicating whether atrial fibrillation is taking place, on the basis of the detection result.

### BACKGROUND

1. Technical Field

The present invention relates to a technique of deciding presence of atrial fibrillation.

2. Related Art

In the medical field related to heart diseases, techniques of deciding the presence of atrial fibrillation are employed. For example, JP-A-2009-89883 discloses a technique of measuring an RR interval of each pulse obtained from an electrocardiogram and deciding whether atrial fibrillation is taking place on the basis of standard deviation and frequency distribution of the RR intervals. According to “Spectral characteristics of ventricular response to atrial fibrillation” by Hayano J, Yamasaki F, Sakata S, Okada A, Mukai S, and Fujinami T, in American Journal of Physiology 1997; 273: H2811-H2816 (hereinafter, non-patent document), the atrial fibrillation presents irregular RR intervals, and 1/f β components are found through frequency analysis of pulses of the atrial fibrillation and fluctuation of the 1/f β components produces white noise.

To accurately detect the atrial fibrillation with the techniques according to JP-A-2009-89883 and the non-patent document, the RR interval of each pulse has to be accurately measured. The RR interval can be measured on the basis of a waveform signal of the cardiogram obtained through electrocardiography, and also on the basis of a pulse wave signal obtained through measurement of pulse wave.

In the measurement of the pulse wave, however, the subject is often free to move around during the measurement, and therefore body motion noise is prone to affect the pulse wave signal. In the case of electrocardiography also, the body motion noise may affect the waveform signal of the electrocardiogram, though the impact may be relatively smaller. The body motion noise significantly degrades the measurement accuracy of the RR interval of each pulse.

Consequently, in the case where the accurate measurement of the RR interval of each pulse is the essential requirement as in the techniques according to JP-A-2009-89883 and the non-patent document, it is undesirable to utilize the signal affected by the body motion noise for making decision on whether atrial fibrillation is taking place.

### SUMMARY

Accordingly, an advantage of some aspects of the invention is that a technique is provided that enables the presence of atrial fibrillation to be decided on the basis of a signal that allows measurement of RR intervals, such as a pulse wave signal and a waveform signal of an electrocardiogram, despite the signal being affected by body motion noise.

In an aspect, the invention provides an atrial fibrillation decision apparatus including an acquisition unit that acquires a detected waveform signal indicating a detection result of an electrocardiogram or pulse wave, an RR interval calculation unit that calculates, with respect to each of a plurality of frames, a parameter corresponding to an average RR interval of the frame on the basis of a spectrum of the frame obtained through frequency analysis of the detected waveform signal acquired, a power calculation unit that calculates a power of a predetermined frequency band in an RR waveform signal indicating a temporal change of the average RR interval calculated by the RR interval calculation unit, and a decision unit that decides whether the calculated power satisfies a specific condition and outputs information indicating whether atrial fibrillation is taking place, on the basis of the detection result.

The atrial fibrillation decision apparatus thus configured enables the presence of atrial fibrillation to be decided on the basis of the signal that allows measurement of RR intervals, such as a pulse wave signal and a waveform signal of an electrocardiogram, despite the signal being affected by body motion noise.

Preferably, the atrial fibrillation decision apparatus may further include a fluctuation coefficient calculation unit that calculates a fluctuation coefficient of the average RR interval in the RR waveform signal, and the decision unit may decide whether a set of the power and the fluctuation coefficient satisfies the specific condition, and output the information indicating whether atrial fibrillation is taking place in accordance with the decision result.

Preferably, the decision unit may divide a plurality of sets of the power and the fluctuation coefficient acquired from a plurality of frames into a first cluster of a relatively high power and high fluctuation coefficient and a second cluster of a relatively low power and low fluctuation coefficient, and decide whether atrial fibrillation is taking place on the basis of a positional relationship between a first center of gravity of the first cluster and a second center of gravity of the second cluster in a fluctuation coefficient-power coordinate.

The atrial fibrillation decision apparatus thus configured enables the decision on whether atrial fibrillation is taking place to be made by using the average RR interval.

Preferably, the decision unit may decide the first cluster as representing a state where atrial fibrillation is taking place, in the case where the first center of gravity and the second center of gravity are spaced from each other by a distance equal to or larger than a first predetermined threshold.

The atrial fibrillation decision apparatus thus configured enables the decision on whether atrial fibrillation is taking place to be made on the basis of a clustering result.

Preferably, the decision unit may decide the first cluster and the second cluster as representing a state where atrial fibrillation is taking place, in the case where the spacing between the first center of gravity and the second center of gravity is smaller than the first predetermined threshold, and the average center of gravity of the first center of gravity and the second center of gravity has a power equal to or larger than a second predetermined threshold and a fluctuation coefficient equal to or higher than a third predetermined threshold.

The atrial fibrillation decision apparatus thus configured enables the decision on whether atrial fibrillation is taking place to be made, even when the first cluster and the second cluster are close to each other.

Preferably, the decision unit may decide the first cluster and the second cluster as not representing a state where atrial fibrillation is taking place, in the case where the spacing between the first center of gravity and the second center of gravity is smaller than the first predetermined threshold, and the average center of gravity of the first center of gravity and the second center of gravity does not have either or both of a power equal to or larger than the second predetermined threshold and a fluctuation coefficient equal to or higher than the third predetermined threshold.

The atrial fibrillation decision apparatus thus configured enables the decision on whether atrial fibrillation is taking place to be made, even when the first cluster and the second cluster are close to each other.

Preferably, a minimum frequency of the frequency band may be equal to or higher than the reciprocal of the time of the frame.

Such an arrangement contributes to improving the decision accuracy of the atrial fibrillation.

Preferably, the atrial fibrillation decision apparatus may further include a detection unit that detects the electrocardiogram or the pulse wave of the subject, and a notification unit that notifies the subject of the information outputted by the decision unit, and the acquisition unit may acquire the detected waveform signal obtained on the basis of the detection result.

Such a configuration allows the subject to confirm in real time the decision result about the atrial fibrillation.

Preferably, the acquisition unit may include a noise reduction unit that filters out components originating from body motion noise from the detected waveform signal thus reducing the noise, and outputs the filtered signal as the detected waveform signal.

Such a configuration allows the subject to confirm in real time the decision result about the atrial fibrillation, with improved decision accuracy of the atrial fibrillation.

In another aspect, the invention provides a method of deciding presence of atrial fibrillation including acquiring a detected waveform signal indicating a detection result of an electrocardiogram or pulse wave, calculating, with respect to each of a plurality of frames, a parameter corresponding to an average RR interval of the frame on the basis of a spectrum of the frame obtained through frequency analysis of the detected waveform signal acquired, calculating a power of a predetermined frequency band in an RR waveform signal indicating a temporal change of the calculated average RR interval, calculating a fluctuation coefficient of the average RR interval in the RR waveform signal, deciding whether a set of the power and the fluctuation coefficient satisfies a specific condition, and outputting information indicating whether atrial fibrillation is taking place, on the basis of the decision result.

The decision method thus arranged enables the decision on whether atrial fibrillation is taking place to be made on the basis of the signal that allows measurement of RR intervals, such as a pulse wave signal and a waveform signal of an electrocardiogram, despite the signal being affected by body motion noise.

In still another aspect, the invention provides a program that causes a computer to acquire a detected waveform signal indicating a detection result of an electrocardiogram or pulse wave, calculate, with respect to each of a plurality of frames, a parameter corresponding to an average RR interval of the frame on the basis of a spectrum of the frame obtained through frequency analysis of the detected waveform signal acquired, calculate a power of a predetermined frequency band in an RR waveform signal indicating a temporal change of the calculated average RR interval, calculate a fluctuation coefficient of the average RR interval in the RR waveform signal, decide whether a set of the power and the fluctuation coefficient satisfies a specific condition, and output information indicating whether atrial fibrillation is taking place, on the basis of the decision result.

The program thus arranged enables the decision on whether atrial fibrillation is taking place to be made on the basis of the signal that allows measurement of RR intervals, such as a pulse wave signal and a waveform signal of an electrocardiogram, despite the signal being affected by body motion noise.

### BRIEF DESCRIPTION OF THE DRAWINGS

The invention will be described with reference to the accompanying drawings, wherein like numbers reference like elements.

FIGS. 1A and 1B are a perspective view and a fragmentary side view respectively, showing an appearance of a pulse wave measurement device according to an embodiment of the invention.

FIG. 2 is a block diagram showing a configuration of the pulse wave measurement device according to the embodiment.

FIG. 3 is a block diagram showing a functional configuration of an atrial fibrillation decision apparatus according to the embodiment.

FIG. 4 is a diagram for explaining a frame for performing frequency analysis of a detected waveform signal.

FIG. 5 is a diagram for explaining frequency characteristics in an RR interval calculation unit.

FIG. 6 is a diagram for explaining a frame for performing frequency analysis of an RR waveform signal.

FIG. 7 is a graph for explaining a period during which atrial fibrillation was detected.

FIG. 8 is a flowchart showing an atrial fibrillation decision process according to the embodiment.

FIGS. 9A and 9B are graphs for explaining the atrial fibrillation decision method based on electrocardiographic RR intervals.

FIGS. 10A and 10B are graphs for explaining the drawback of the atrial fibrillation decision method based on an average pulse wave RR interval.

FIG. 11 is a flowchart showing the detailed process of the atrial fibrillation decision at step S410 in FIG. 8.

FIG. 12 shows exemplary graphs respectively indicating a power waveform signal Pa and a fluctuation coefficient signal Sc.

FIG. 13 shows exemplary graphs respectively indicating the power and the fluctuation coefficient, subjected to a moving average process.

FIG. 14 is a graph showing a relationship between the power and the fluctuation coefficient.

FIG. 15 is a graph showing a result of clustering performed on the graph shown in FIG. 14.

### DESCRIPTION OF EXEMPLARY EMBODIMENTS

### Embodiment

### Outline

To decide the presence of atrial fibrillation from an electrocardiogram, conventionally an RR interval of each pulse (electrocardiographic RR interval) has been utilized. In this embodiment, in contrast, pulse wave is employed for deciding whether atrial fibrillation is taking place. Unlike the case of utilizing the electrocardiogram, however, the RR interval of each pulse may fail to be accurately measured by the method of utilizing the pulse wave. Therefore, this embodiment adopts a value indicating the average of the RR interval during a certain period (frame), which will hereinafter be referred to as “average pulse wave RR interval”. Hereunder, a method of detecting atrial fibrillation by using the electrocardiographic RR interval will first be described, followed by description of a drawback incidental to the method of utilizing the average pulse wave RR interval, and then the outline of the decision method according to this embodiment will be described.

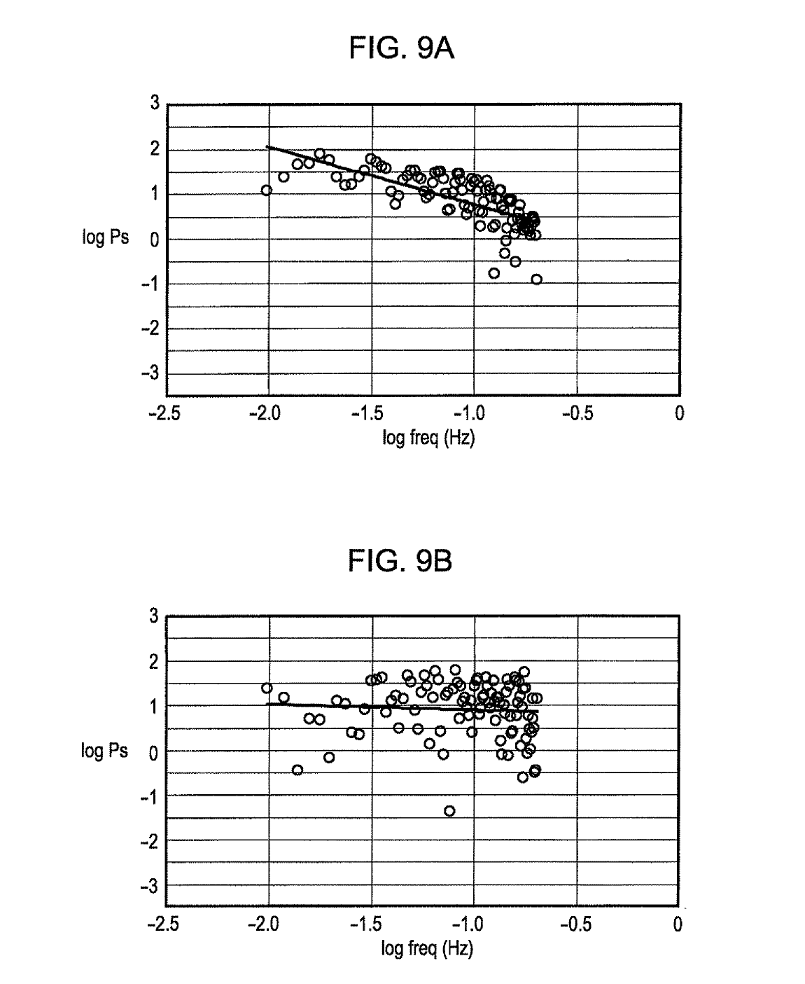

FIGS. 9A and 9B are graphs for explaining the atrial fibrillation detection method that utilizes the electrocardiographic RR interval. FIGS. 9A and 9B represent logarithmically transformed peak frequencies and powers obtained through frequency analysis of waveform signals indicating fluctuation of RR intervals in electrocardiogram, in a frequency band of 0.01 Hz to 0.2 Hz with respect to frames of 480 seconds each. FIG. 9A represents the data based on the RR intervals in electrocardiogram free from atrial fibrillation, and FIG. 9B represents the data based on the RR intervals in electrocardiogram with atrial fibrillation. Lines in FIGS. 9A and 9B represent linear regression obtained from the plotted data. The inclination of the regression line 13 and the correlation coefficient γ calculated on the basis of those graphs are as follows.

In the case of FIG. 9A where atrial fibrillation is not taking place, γ is −0.72 and β is −1.29. In contrast, in the case of FIG. 9B where atrial fibrillation is taking place, γ is −0.07 and β is −0.13. It is understood from the above that in the case where atrial fibrillation is taking place the correlation is lost and white noise appears, and the inclination β becomes close to zero. Thus, the presence of atrial fibrillation can be decided on the basis of the inclination β of the linear regression line and the correlation coefficient γ obtained from the plotted data of the peak frequency and power, in the case of utilizing the electrocardiographic RR interval.

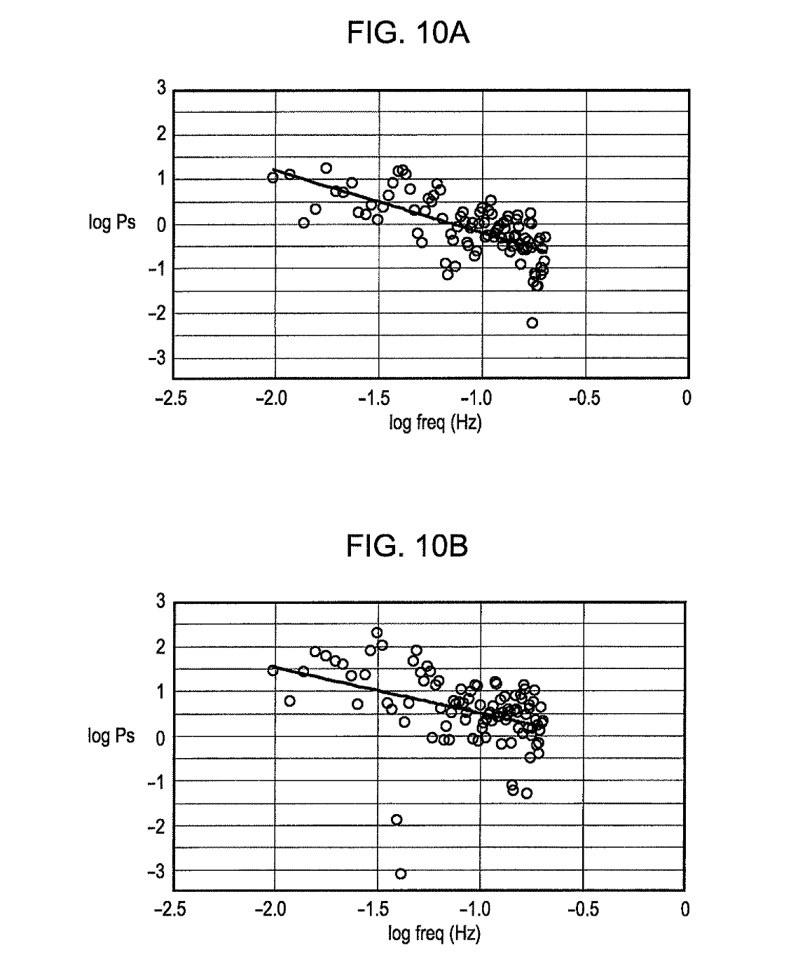

FIGS. 10A and 10B are graphs for explaining the drawback of the atrial fibrillation decision method based on the average pulse wave RR interval. FIGS. 10A and 10B also represent logarithmically transformed peak frequencies and powers obtained through frequency analysis of waveform signals indicating fluctuation of RR intervals in electrocardiogram, in a frequency band of 0.01 Hz to 0.2 Hz with respect to frames of 480 seconds each. FIG. 10A represents the data based on the RR intervals in electrocardiogram free from atrial fibrillation, and FIG. 10B represents the data based on the RR intervals in electrocardiogram with atrial fibrillation. Lines in FIGS. 10A and 10B represent linear regression obtained from the plotted data.

In the case of FIG. 10A where atrial fibrillation is not taking place, γ is −0.68 and β is −1.40. In contrast, in the case of FIG. 10B where atrial fibrillation is taking place, γ is −0.41 and β is −1.02. Thus, in the case of utilizing the average pulse wave RR interval, a significant difference in γ and β is not observed between whether atrial fibrillation is taking place or not as shown in FIGS. 10A and 10B, which makes it difficult to decide the presence of atrial fibrillation through the same process employed in the case of utilizing the electrocardiographic RR interval.

Through comparison of FIGS. 9A and 9B, it is understood that the power increases on the higher frequency side in the case where atrial fibrillation is taking place. For example, through comparison of the power in the frequency band close to 0.2 Hz, the power free from atrial fibrillation is 1.59, and the power with atrial fibrillation is 4.97. Thus, between the cases where atrial fibrillation is taking place and not taking place, the power of the same frequency band increases to several times, which may be construed as a significant difference.

Such an increase in power is also observed in the case of utilizing the average pulse wave RR interval. In FIGS. 10A and 10B, the power free from atrial fibrillation is 0.05, and the power with atrial fibrillation is 0.30 in the frequency band close to 0.2 Hz. Thus, in the case of utilizing the average pulse wave RR interval also, the power of the same frequency band increases to several times, which may be construed as a significant difference, between the cases where atrial fibrillation is taking place and not taking place. In this embodiment, therefore, the increase in power is utilized as an index for deciding whether atrial fibrillation is taking place.

The fluctuation coefficient is another index for deciding whether atrial fibrillation is taking place employed in this embodiment. The fluctuation coefficient is a parameter that indicates an extent of fluctuation of the RR intervals constituting the average pulse wave RR interval, with respect to the average. When atrial fibrillation takes place, the RR interval becomes arrhythmic, in other words the temporal interval of each pulse becomes irregular. This also applies to the average pulse wave RR interval, and therefore the arrhythmicity (fluctuation with respect to the average) may be adopted as an index of atrial fibrillation. In this embodiment, the power and the fluctuation coefficient are employed as indices for deciding whether atrial fibrillation is taking place. Hereafter, the structure and operation according to this embodiment will be described in details.

### Configuration of Pulse Wave Measurement Device 1

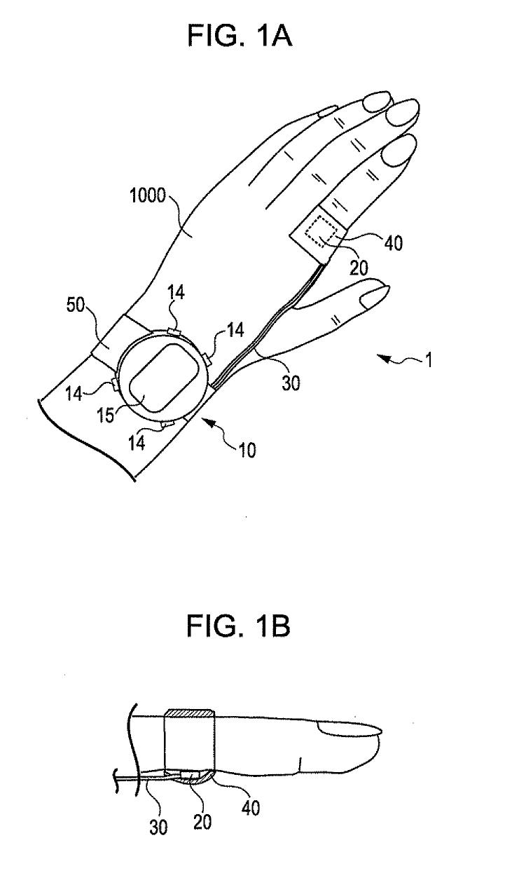

FIGS. 1A and 1B are a perspective view and a fragmentary side view respectively, showing an appearance of a pulse wave measurement device 1 according to an embodiment of the invention. The pulse wave measurement device 1 according to the embodiment includes, as shown in FIG. 1A, a main unit 10 attached like a wrist watch to a wrist (hand) 1000 of a user, i.e., the subject of atrial fibrillation decision, and a pulse wave detection unit 20 attached to a detecting position for detecting a pulse wave. The main unit 10 and the pulse wave detection unit 20 connected to each other via a cable 30. The cable 30 is used to provide a pulse wave signal outputted by the pulse wave detection unit 20 (hereinafter, detected waveform signal L) to the main unit 10, and to supply power from the main unit 10 to the pulse wave detection unit 20.

A wrist strap 50 is attached to the main unit 10. The main unit 10 is attached to the hand of the user upon winding the wrist strap 50 around the wrist of the user. The main unit 10 includes an operation unit 14 and a display unit 15. The operation unit 14 includes operating elements such as button switches for the user to input, for example, a selected function to the pulse wave measurement device 1. The operation unit 14 may also include a touch sensor incorporated in the display unit 15. The display unit 15 includes a display device such as an LCD or an organic EL display.

In this embodiment, the detecting position where the pulse wave detection unit 20 is attached is between the base portion and the PIP joint of the index finger of the hand 1000 as shown in FIG. 113, however the pulse wave detection unit 20 may be attached to any position provided that the pulse wave can be detected. The pulse wave detection unit 20 is fixed to the detecting position by means of a fixing strap 40. The fixing strap 40 is arranged so as to cover the pulse wave detection unit 20, thus restricting a light from outside the fixing strap 40 from reaching a photodetector of the pulse wave detection unit 20.

The pulse wave detection unit 20 detects the pulse wave and outputs the detected waveform signal L indicating the detection result as described below. The pulse wave detection unit 20 includes a light emitter (for example, a green light emitting diode (LED)) and the photodetector. The pulse wave detection unit 20 causes the light emitter to emit a light corresponding to the power supplied by the main unit 10 through the cable 30. Then the pulse wave detection unit 20 receives through the photodetector a portion of the light from the light emitter reflected by hemoglobin in capillary vessels, and provides a signal corresponding to the level of the reflected light to the main unit 10 through the cable 30, as the detected waveform signal L.

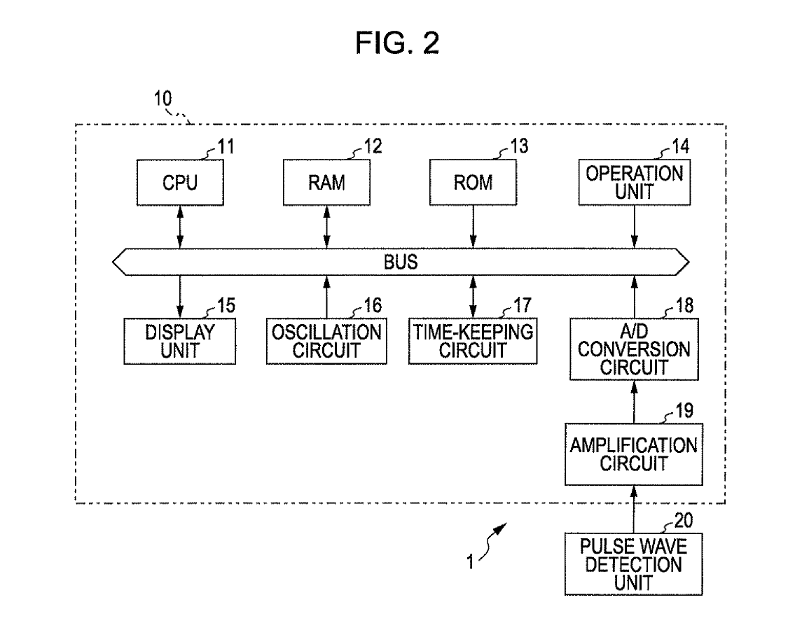

FIG. 2 is a block diagram showing a configuration of the pulse wave measurement device 1 according to the embodiment. The pulse wave measurement device 1 includes the main unit 10 and the pulse wave detection unit 20. The main unit 10 includes a central processing unit (CPU) 11, a random access memory (RAM) 12, a read-only memory (ROM) 13, an operation unit 14, a display unit 15, an oscillation circuit 16, a time-keeping circuit 17, an A/D conversion circuit 18, and an amplification circuit 19. These constituents are connected therebetween via a bus, except for the amplification circuit 19 and the pulse wave detection unit 20.

The CPU 11 performs the control of the constituents and data transfer, in accordance with a control program stored in the ROM 13. The RAM 12 temporarily stores therein physiological information such as the detected waveform signal L, and various data arising during the execution of the control program by the CPU 11. The CPU 11 carries out the atrial fibrillation decision by executing the control program, to thereby allow the pulse wave measurement device 1 to function as an atrial fibrillation decision apparatus. The CPU 11 may be configured to perform various functions in addition to the atrial fibrillation decision, through the execution of the control program. Those functions may be realized, for example, through manipulation of the operation unit 14 by the user.

As stated above, the operation unit 14 includes button switches by which the user can input instructions to the pulse wave measurement device 1. Upon being operated by the user, the operation unit 14 outputs an operational signal to the CPU 11.

The display unit 15 includes a display device such as an LCD or an organic EL display as stated above, and the content to be displayed is controlled by the CPU 11. The display unit 15 displays various images showing a time of the day, program menus, pulse wave measurement result, atrial fibrillation decision result, and so forth.

The oscillation circuit 16 provides a clock signal constituting the base of the control function, to the CPU 11.

The time-keeping circuit 17 measures time under the control of the CPU 11.

The amplification circuit 19 amplifies the detected waveform signal L provided from the pulse wave detection unit 20 through the cable 30. The amplification gain is determined by the CPU 11.

The A/D conversion circuit 18 converts the detected waveform signal L amplified by the amplification circuit 19, which is an analog signal, to a digital signal. In this embodiment, the sampling frequency is set at 100 Hz, which is sufficiently higher than the RR interval obtained from the pulse wave. The quantization bit rate is 10 bits in this embodiment. However, the sampling frequency and the quantization bit rate may be set at different values in accordance with the required accuracy level.

Hereunder, the functional configuration (atrial fibrillation deciding function) of the atrial fibrillation decision apparatus, realized by the CPU 11, will be described.

### Functional Configuration

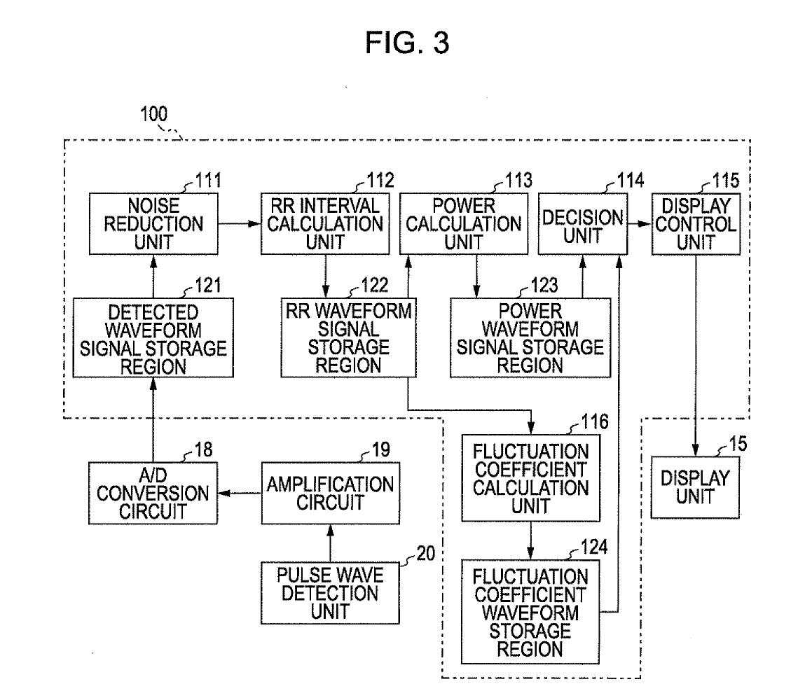

FIG. 3 is a block diagram showing a functional configuration of the atrial fibrillation decision apparatus 100 according to the embodiment. The atrial fibrillation decision apparatus 100 includes a noise reduction unit 111, an RR interval calculation unit 112, a power calculation unit 113, a decision unit 114, a display control unit 115, and a fluctuation coefficient calculation unit 116, as well as storage regions for various data including a detected waveform signal storage region 121, an RR waveform signal storage region 122, a power waveform signal storage region 123, and a fluctuation coefficient storage region 124.

The detected waveform signal storage region 121 is a region on the RAM 12 allocated for the detected waveform signal L converted into a digital signal by the A/D conversion circuit 18.

The noise reduction unit 111 filters out body motion noise components of frequencies deviating from the frequency band corresponding to the RR interval from the detected waveform signal L stored in the detected waveform signal storage region 121, and outputs the filtered signal. Examples of the applicable filter include a high-pass filter, a band-pass filter, and an adaptive filter. The detected waveform signal L that has undergone the body motion noise reduction by the noise reduction unit 111 may be once stored in the RAM 12. The detected waveform signal storage region 121 and the noise reduction unit 111 serve as the acquisition unit that acquires the detected waveform signal L employed for frequency analysis in the RR interval calculation unit 112.

Here, although the body motion noise components are reduced through the foregoing process and hence the detected waveform signal L is alleviated from the impact thereof, the extent of the noise reduction is not yet sufficient to allow accurate measurement of the RR interval for performing reliable atrial fibrillation decision with the technique according to the related art (JP-A-2009-89883 and the non-patent document).

The RR interval calculation unit 112 cuts out frames for each sampling process from the detected waveform signal L from which the body motion noise components are reduced by the noise reduction unit 111, and calculates a frequency spectrum through a short-time frequency analysis (short-time Fourier transform (STFT)). Then the RR interval calculation unit 112 calculates a parameter corresponding to the RR interval with respect to each frame on the basis of the calculated frequency spectrum, and stores an RR waveform signal FRR indicating the temporal change of the parameter in the RR waveform signal storage region 122 provided on the RAM 12. Here, the RR waveform signal FRR is a set of data indicating the temporal change of the parameter.

The parameter thus calculated indicates the average of RR intervals in the pulse wave in each frame in this embodiment and is, for example, a frequency corresponding to the highest peak of the frequency spectrum. Accordingly, the RR waveform signal FRR indicates the temporal change of the average RR interval in pulse wave. Such a process performed by the RR interval calculation unit 112 significantly suppresses the impact of the body motion noise contained in the RR waveform signal FRR, despite that the body motion noise is not fully removed by the noise reduction unit 111.

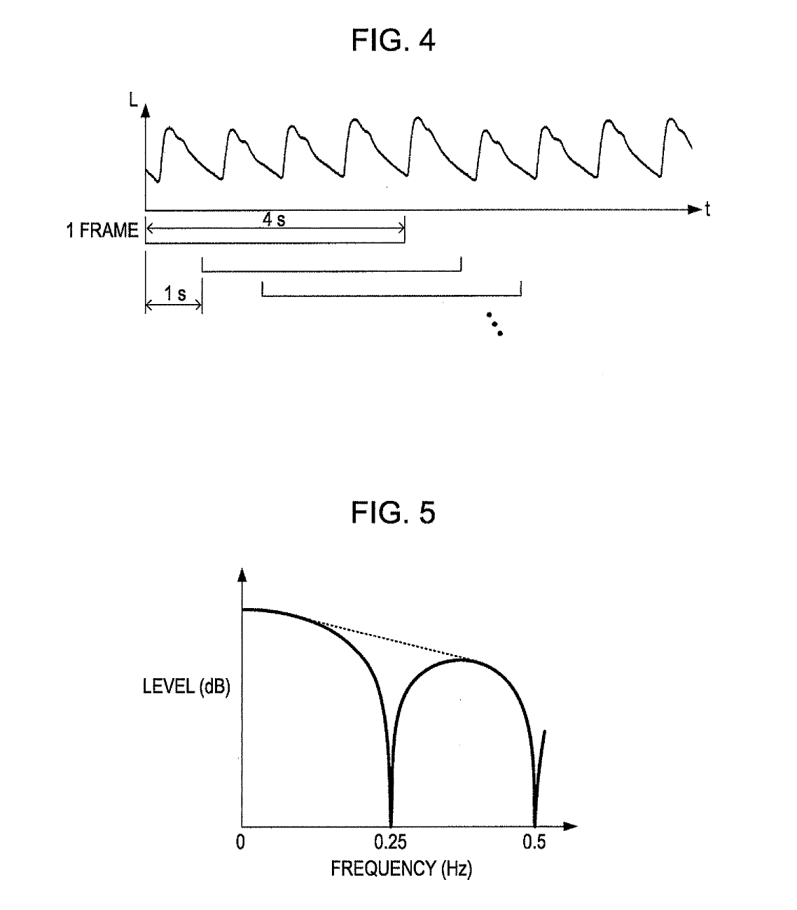

FIG. 4 is a diagram for explaining the frame for performing the frequency analysis of the detected waveform signal L. The waveform shown in FIG. 4 represents that of the detected waveform signal L. As shown in FIG. 4, the period of each frame is 4 seconds in this embodiment, and sampling is performed every second for performing the frequency analysis. Accordingly, the frames are defined with a delay of one second one after another, and overlap with the subsequent one over a period of 3 seconds. With such a setting of the sampling timing and the frames, the average RR interval in pulse wave is taken from the RR intervals in 4 seconds, and the RR waveform signal FRR indicates every second the variation of the average RR interval in pulse wave.

FIG. 5 is a diagram for explaining frequency characteristics in the RR interval calculation unit 112. In the RR interval calculation unit 112, performing the frequency analysis on the basis of the frames defined as above is equivalent to superposing the frequency characteristics through moving average calculation. The frequency characteristics shown in FIG. 5 present a valley at the frequency of 0.25 Hz, corresponding to 4 seconds which is the time of the frame, and integer times thereof, with a general tendency defined by connecting the apices of the hills that the level becomes lower at a higher frequency, i.e., with a negative inclination. The longer the time of the frame is, the steeper the inclination becomes. Conversely, although the inclination becomes closer to zero by setting a shorter time of the frame, residual body motion noise components increase in the detected waveform signal L. Accordingly, it is preferable to set the time of the frame between 1 second and 5 seconds, more preferably between 2 seconds and 4 seconds.

The power calculation unit 113 performs the STFT analysis with respect to the RR waveform signal FRR stored in the RR waveform signal storage region 122, to thereby calculate the power (band power) of a part of the frequency bands (calculation frequency band) on the basis of the frequency spectrum obtained. The power calculation unit 113 then stores a power waveform signal Pa indicating the temporal change of the calculated band power, in the power waveform signal storage region 123 provided on the RAM 12. Here, the power waveform signal Pa is a set of data indicating the temporal change of the band power.

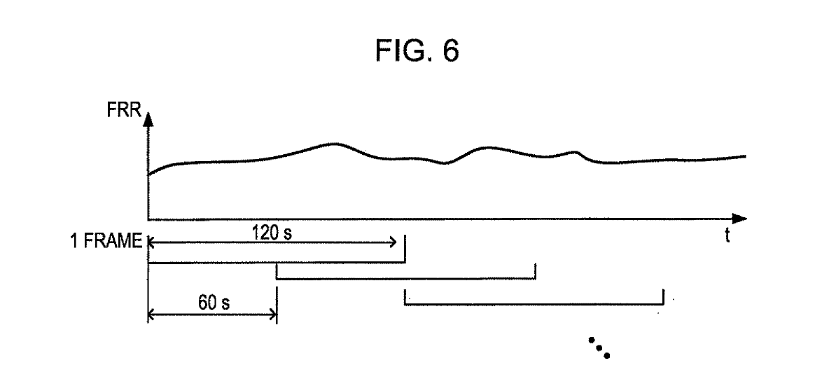

FIG. 6 is a diagram for explaining the frame for performing frequency analysis of the RR waveform signal FRR. The waveform shown in FIG. 6 is an example of that of the RR waveform signal FRR. As shown therein, the time of each frame is 120 seconds in this example, and sampling is performed every 60 seconds for frequency analysis. In other words, each frame is defined with a delay of 60 seconds on after another, and overlaps with the subsequent frame over a period of 60 seconds.

In addition, the calculation frequency band at which the power calculation unit 113 calculates the band power is determined in advance, which corresponds to the band between 0.25 Hz and 0.5 Hz in this example. This corresponds to the band between the two valleys (at 0.25 Hz and 0.5 Hz) of the frequency characteristics shown in FIG. 5. This is because the power corresponding to the valley is suppressed and hence barely involved in the decision of atrial fibrillation, and therefore the calculation frequency band is determined focusing on the portion that effectively serves for the decision of atrial fibrillation. Accordingly, the calculation frequency band may be determined by further deducting the portion corresponding to the valleys of the frequency characteristics, such as between 0.3 Hz and 0.45 Hz, so that only the portion corresponding to the hill is involved.

Here, a minimum frequency (lower limit) and a maximum frequency (upper limit) of the calculation frequency band are determined, in this embodiment, in accordance with the RR frequency characteristics in the interval calculation unit 112, i.e., the time of the frame employed for the frequency analysis by the RR interval calculation unit 112. However, one or both of the upper and lower limits of the frequency do not have to be determined in accordance with the time of the frame.

Preferably, the minimum frequency of the calculation frequency band is, as shown in FIGS. 9A, 9B, 10A, and 10B, equal to or higher than 0.1 Hz, and more preferably equal to or higher than 0.2 Hz, where the variation of the power can be clearly observed. Further, it is still more preferable that the minimum frequency is, as stated earlier, equal to or higher than the reciprocal of the time of the frame used for the frequency analysis by the RR interval calculation unit 112.

In addition, it is preferable that the maximum frequency of the calculation frequency band is equal to or lower than ½ of the sampling frequency for the frequency analysis by the RR interval calculation unit 112, from the viewpoint of influence of the Nyquist frequency. Further, it is more preferable that the maximum frequency is, as stated earlier, equal to or lower than twice of the reciprocal of the time of the frame used for the frequency analysis by the RR interval calculation unit 112.

Referring again to FIG. 3, the fluctuation coefficient calculation unit 116 calculates the fluctuation coefficient CVRR on the basis of the RR waveform signal FRR (average pulse wave RR interval) stored in the RR waveform signal storage region 122, in accordance with the following equation (1).

CVRR=σRR/aveRR (1)

Here, the terms σRR and aveRR represent the standard deviation and the average, respectively, of the average pulse wave RR interval of a period of 1 frame. Accordingly, the fluctuation coefficient CVRR is a parameter indicating an extent of fluctuation with respect to the average. The fluctuation coefficient calculation unit 116 stores the fluctuation coefficient CVRR thus calculated in the fluctuation coefficient waveform storage region 124. Since the fluctuation coefficient CVRR is calculated with respect to each frame, the signal indicating the temporal change of the fluctuation coefficient CVRR (hereinafter, fluctuation coefficient signal Sc) is stored in the fluctuation coefficient waveform storage region 124. Here, the fluctuation coefficient signal Sc is a set of data indicating the temporal change of the fluctuation coefficient CVRR.

The decision unit 114 decides whether the power waveform signal Pa stored in the power waveform signal storage region 123 and the fluctuation coefficient signal Sc stored in the fluctuation coefficient waveform storage region 124 satisfy a specific criterion, and outputs information reflecting the decision result. The specific criterion will be subsequently described.

The decision unit 114 outputs, upon deciding that atrial fibrillation is taking place, the information indicating such a decision result to the display control unit 115. The information outputted by the decision unit 114 may be of any form provided that the information indicates whether atrial fibrillation is taking place, for example information indicating whether atrial fibrillation has been observed. The information display control unit 115 controls the content to be displayed on the display unit 15 on the basis of the information outputted by the decision unit 114, thereby causing the display unit 15 to display an image showing that atrial fibrillation has been decided. In view of such a displayed content, the user can confirm whether atrial fibrillation is taking place. Here, the display may indicate a real time decision of the atrial fibrillation, or the period during which it has been decided that atrial fibrillation is taking place.

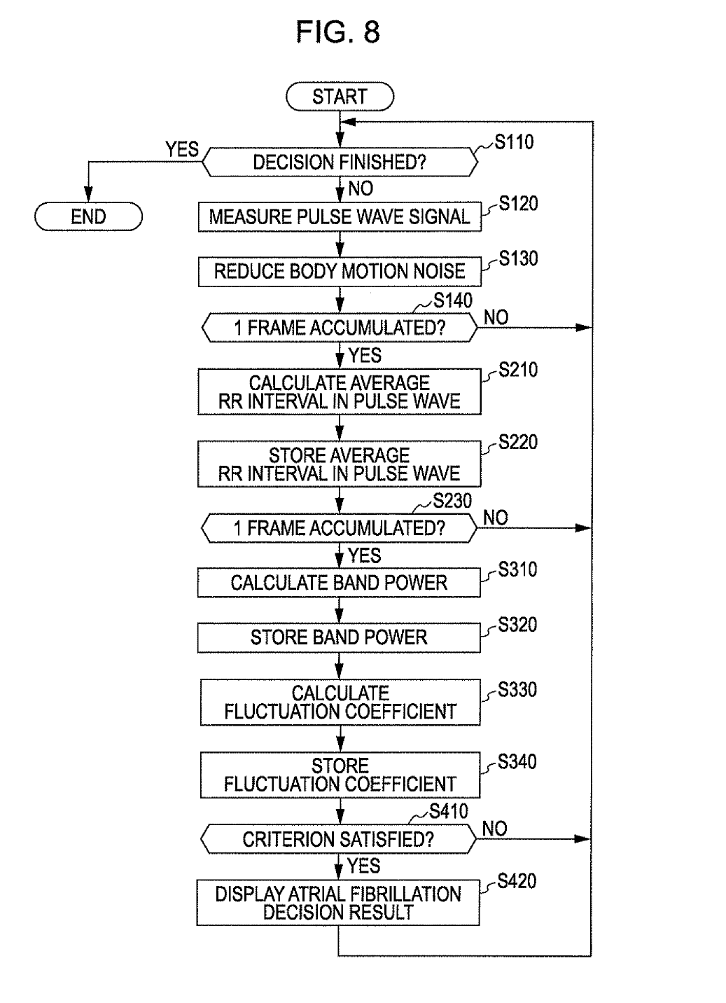

The functional configuration of the atrial fibrillation decision apparatus 100 is as above. Now, an operation of the atrial fibrillation decision apparatus 100 (atrial fibrillation decision process) will be described referring to FIG. 8.

### Atrial Fibrillation Decision Process

FIG. 8 is a flowchart showing the atrial fibrillation decision process according to the embodiment. Once the user operates the operation unit 14 so as to input the instruction to start the atrial fibrillation decision process, the CPU 11 starts the process shown in FIG. 8. First, the CPU 11 decides whether the user has inputted through the operation unit 14 an instruction to finish the decision process (step S110). In the case where the instruction to finish the decision process has been inputted (YES at step S110), the CPU 11 finishes the atrial fibrillation decision process.

In the case where the instruction to finish the decision process has not been inputted (NO at step S110), the CPU 11 causes the pulse wave detection unit 20 to detect the pulse wave to thereby measure the detected waveform signal L (step S120), and causes the noise reduction unit 111 to reduce the body motion noise (step S130). At this stage the CPU 11 stores the detected waveform signal L in the detected waveform signal storage region 121 on the RAM 12 in this embodiment, however the CPU 11 may store the detected waveform signal L that has undergone the body motion noise reduction.

The CPU 11 then decides whether the waveform signals that have undergone the body motion noise reduction corresponding to one frame have been accumulated in the RAM 12 (step S140). In the case where the waveform signals corresponding to one frame have not been accumulated (NO at step S140), the CPU 11 resumes the operation from the step S110. In contrast, in the case where the waveform signals corresponding to one frame have been accumulated (YES at step S140), the CPU 11 causes the RR interval calculation unit 112 to calculate the average RR interval in pulse wave (step S210).

Then the CPU 11 stores the average RR interval in pulse wave calculated by the RR interval calculation unit 112 in the RR waveform signal storage region 122 (step S220). The temporal change of the average RR interval in pulse wave stored in the storage region constitutes the RR waveform signal FRR.

The CPU 11 decides whether the RR waveform signals FRR corresponding to one frame have been accumulated in the RR waveform signal storage region 122 (step S230). In the case where the RR waveform signals FRR corresponding to one frame have not been accumulated (NO at step S230), the CPU 11 resumes the operation from the step S110. In the case where the RR waveform signals FRR corresponding to one frame have been accumulated (YES at step S230), the CPU 11 causes the power calculation unit 113 to calculate the band power (step S310).

The CPU 11 stores the band power calculated by the power calculation unit 113 in the power waveform signal storage region 123 (step S320). The temporal change of the band power stored in the storage region constitutes the power waveform signal Pa.

The CPU 11 causes the fluctuation coefficient calculation unit 116 to calculate the fluctuation coefficient (step S330). The CPU 11 stores the fluctuation coefficient calculated by the fluctuation coefficient calculation unit 116 in the fluctuation coefficient waveform storage region 124 (step S340).

Then the CPU 11 looks up the power waveform signals Pa and the fluctuation coefficient signals Sc thus far stored, and causes the decision unit 114 to decide whether the power waveform signals Pa and the fluctuation coefficient signals Sc satisfy the predetermined criterion for atrial fibrillation has been satisfied, i.e., whether the threshold Pth of the band power has been exceeded for a period equal to or longer than 50% of the last 30 minutes of observation (step S410).

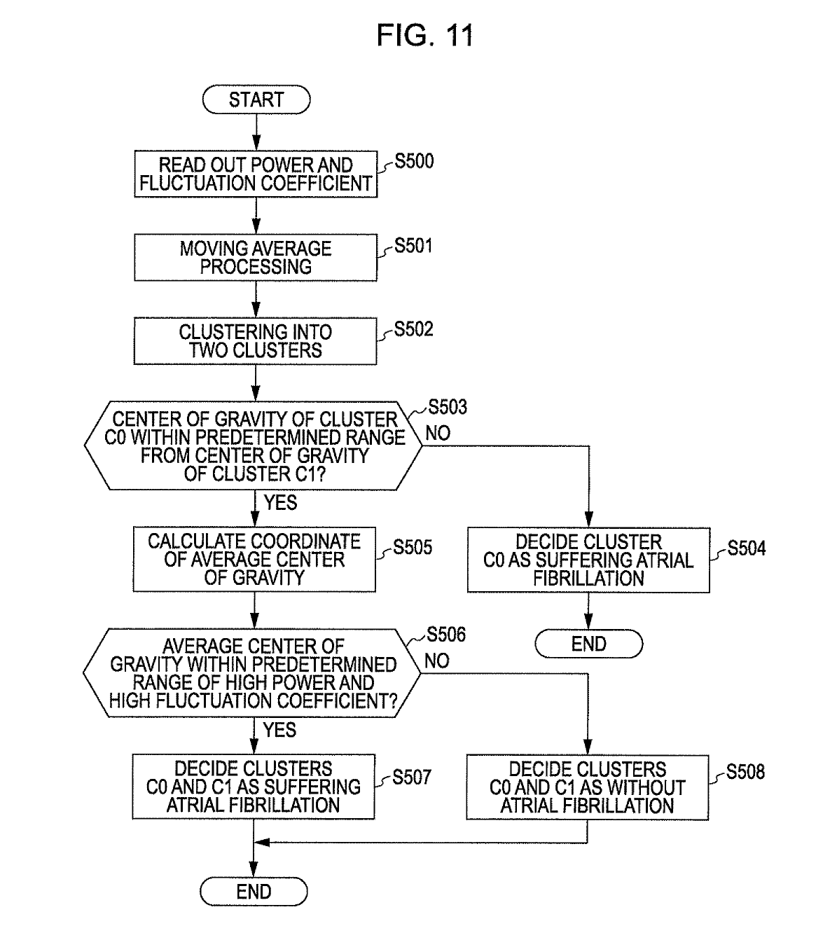

FIG. 11 is a flowchart showing the detailed process of the atrial fibrillation decision at step S410. At step S500, the CPU 11 reads out the power waveform signal Pa and the fluctuation coefficient signal Sc from the RAM 12.

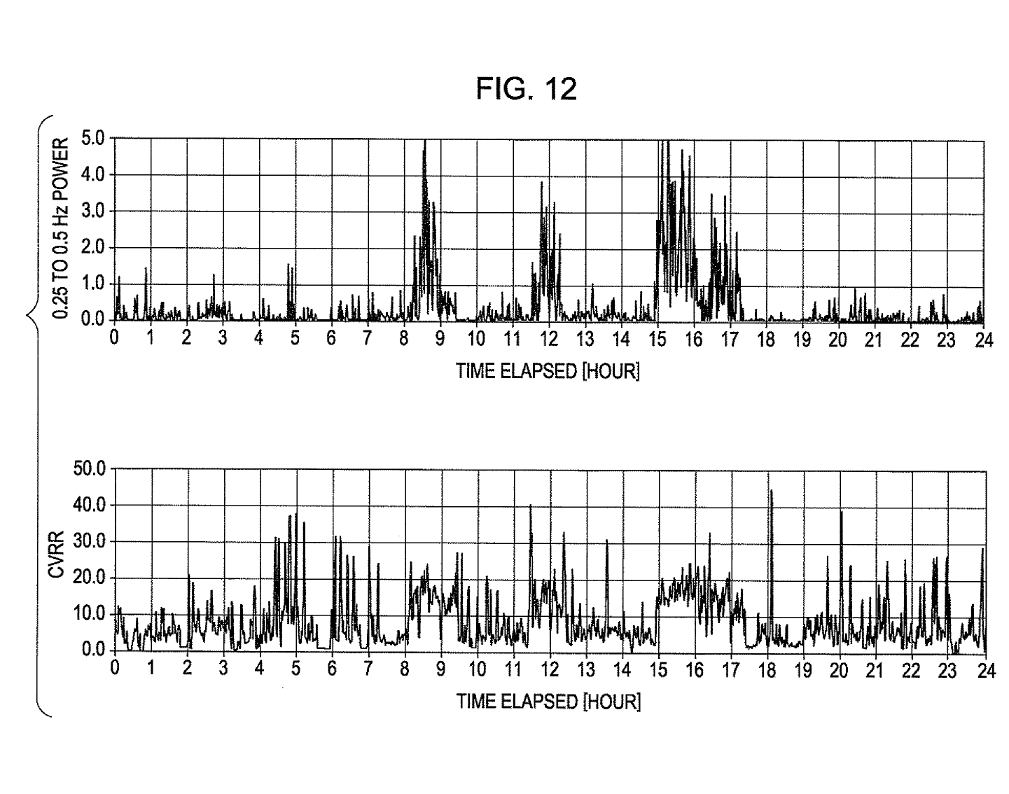

FIG. 12 shows exemplary graphs respectively indicating a power waveform signal Pa and a fluctuation coefficient signal Sc. FIG. 12 shows the power waveform signal Pa (msec²) and the fluctuation coefficient signal Sc (%) obtained from pulse wave signals of a patient measured for a period of 24 hours. It is to be noted that this patient suffered atrial fibrillation during the measurement period.

Referring back to FIG. 11, the CPU 11 applies a moving average processing to the power waveform signal Pa and the fluctuation coefficient signal Sc (step S501). The moving average processing is performed so as to level off fine fluctuations (fluctuation within a short time), with respect to each of the power and the fluctuation coefficient CVRR. In this example, 20 pieces of data, i.e., data obtained from measurement for 20 minutes, are employed for the moving average processing.

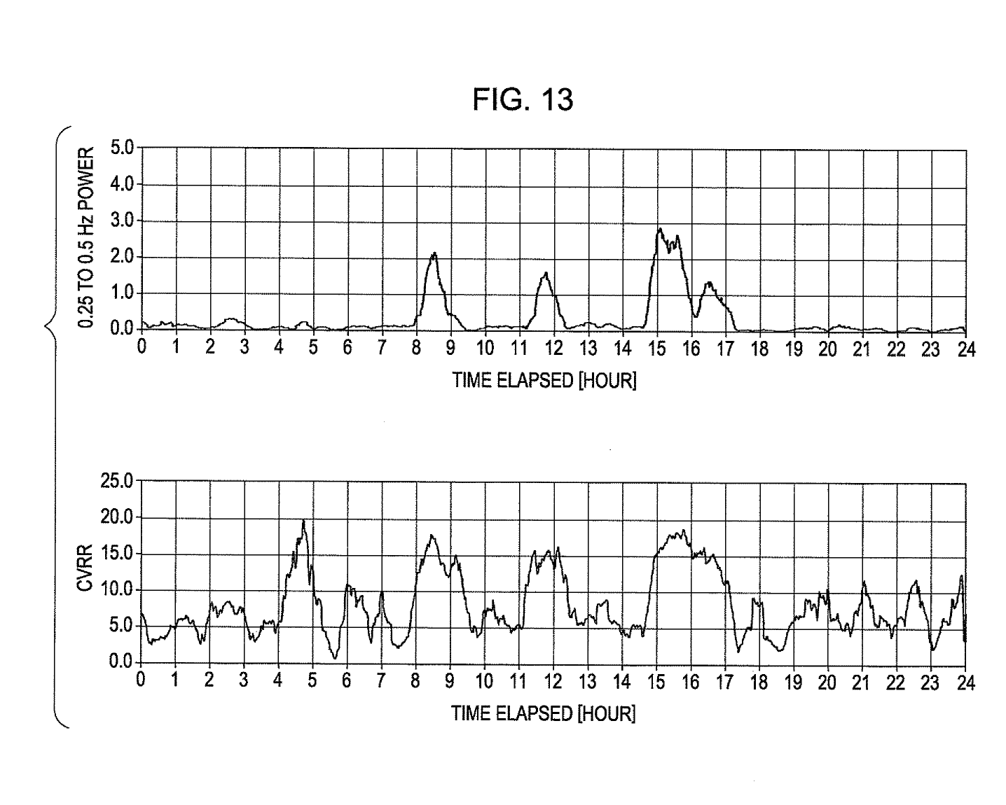

FIG. 13 shows exemplary graphs respectively indicating the power waveform signal Pa and the fluctuation coefficient signal Sc subjected to the moving average processing. Fine fluctuations have been leveled off through the moving average processing. Hereafter, the data after the moving average processing will be construed to indicate the power and the fluctuation coefficient of a certain time of the day. Since the measurement is performed every 60 seconds, 1440 pieces of data are obtained from the measurement for 24 hours.

Referring again to FIG. 11, the CPU 11 divides such data into two clusters by using a predetermined algorithm, such as a k-means process widely known as a clustering method (step S502).

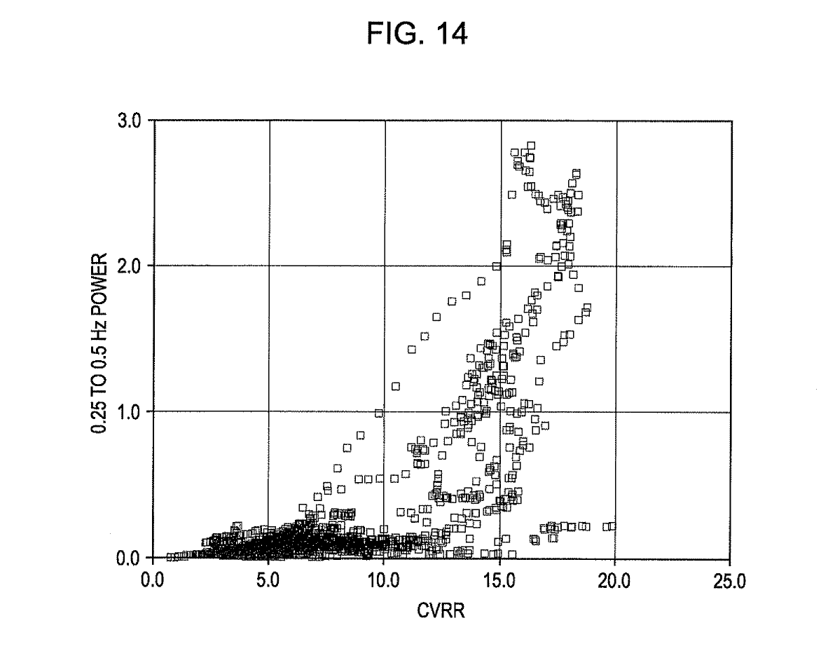

FIG. 14 is a graph showing a relationship between the power and the fluctuation coefficient. The vertical axis represents the power (msec²), and the horizontal axis represents the fluctuation coefficient CVRR (%). As stated above, the power and the fluctuation coefficient become relatively higher when atrial fibrillation takes place, compared with a normal state. Accordingly, it may be assumed that the plots in the upper right region in FIG. 14 correspond to the data measured when atrial fibrillation was taking place. In this embodiment, the data is divided into two clusters by the clustering method, and the decision is made on whether atrial fibrillation is taking place on the basis of the positional relationship between the two clusters in the fluctuation coefficient-power coordinate.

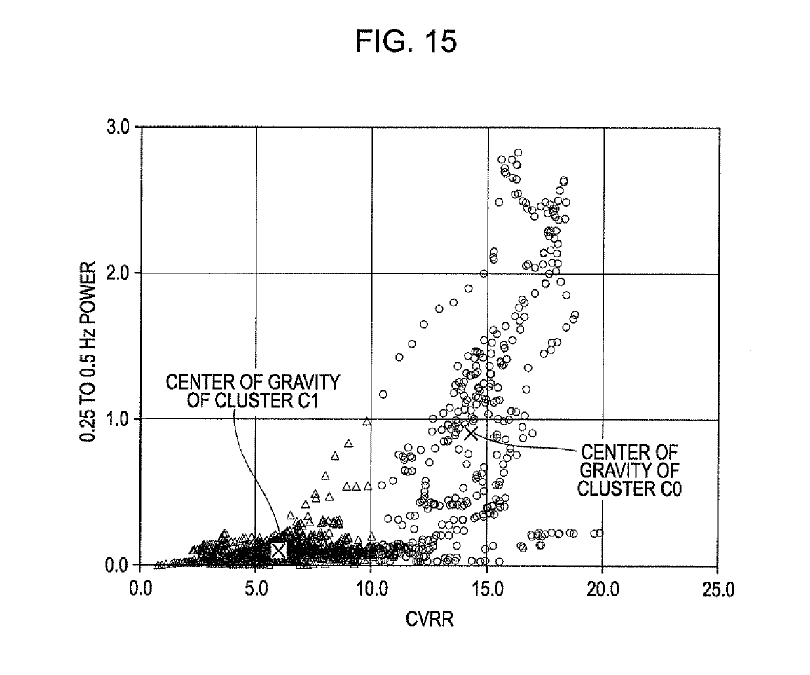

FIG. 15 is a graph showing a result of clustering applied to the data of FIG. 14. The data is thus divided into two clusters by the clustering method (k-means process in this example). The two clusters will hereafter be referred to as cluster C0 and cluster C1, respectively. The cluster C0 represents a data group of relatively high fluctuation coefficient and high power, and the cluster C1 represents a data group of relatively low fluctuation coefficient and low power. The data of the cluster C0 is expressed by blank circles, and the data of the cluster C1 is expressed by triangles. In addition, the position of the center of gravity of the clusters in the fluctuation coefficient-power coordinate can be obtained by the k-means process. FIG. 15 also indicates the respective positions of the center of gravity of the clusters C0 and C1.

Once again, FIG. 11 will be referred to. At step S503, the CPU 11 decides whether the position of the center of gravity of the cluster C0 is within a predetermined range, for example ±30%, with respect to the position of the center of gravity of the cluster C1. In the case where it is decided that the position of the center of gravity of the cluster C0 is outside the range of ±30% from the position of the center of gravity of the cluster C1cluster C0 (NO at S503), the CPU 11 proceeds to step S504. In the case where it is decided that the position of the center of gravity of the cluster C0 is within ±30% from the position of the center of gravity of the cluster C1cluster C0 (YES at S503), the CPU 11 proceeds to step S505.

At step S504, the CPU 11 decides that the cluster C0 represents the data indicating that atrial fibrillation is taking place.

In the case where the position of the center of gravity of the cluster C0 is within ±30% from the position of the center of gravity of the cluster C1, it is decided that the data is unable to be divided into two clusters. Accordingly, as a possibility, it can be assumed that atrial fibrillation has not taken place through the entire measurement period, and also that atrial fibrillation was taking place through the entire measurement period. In this case, the decision of the atrial fibrillation is made depending on the values of the power and fluctuation coefficient. Such a process is performed through steps S505 and thereafter.

At step S505, the CPU 11 calculates the coordinate (hereinafter, coordinate of average center of gravity) of the average position of the center of gravity of the cluster C and that of the cluster C1 (hereinafter, average center of gravity). The average center of gravity may be, for example, a simple average of the center of gravity of the cluster C and that of the cluster C1, in other words the midpoint between the center of gravity of the cluster C and that of the cluster C1. Alternatively, the average center of gravity may be a weighted average of the center of gravity of the cluster C and that of the cluster C1 based on the number of pieces of the data, in other words the center of gravity of all the measurement points.

At step S506, the CPU 11 decides whether the coordinate of average center of gravity is within a predetermined range (for example, 10.0 or higher in fluctuation coefficient and 0.5 or higher in power 0.5). In the case where it is decided that the coordinate of average center of gravity is within the predetermined range (YES at S506), the CPU 11 decides that atrial fibrillation was taking place through the entire measurement period (step 5507). In the case where it is decided that the coordinate of average center of gravity is outside of the predetermined range (NO at S506), the CPU 11 decides that atrial fibrillation has not taken place through the entire measurement period (step S508).

Here, FIG. 8 is again referred to. In the case where it is decided that atrial fibrillation is not taking place (NO at step S410), the CPU 11 resumes the operation from the step S110. In contrast, in the case where it is decided that atrial fibrillation is taking place (YES at step S410), the CPU 11 causes the display control unit 115 to display the decision result that atrial fibrillation is taking place on the display unit 15 (step S420), and then returns to the step S110 to resume the operation.

The CPU 11 may repeatedly perform the process from the step S110 to the step S140, irrespective of the decision made at the step S140. In this case, the CPU 11 may perform the steps S5210 and subsequent thereto in parallel to the process from the step S110 to the step S140, each time the step S140 is decided to be YES. Further, in the case of NO at the step S230 or at the step S410, the CPU 11 may finish the process corresponding to the steps S210 and subsequent thereto performed in parallel.

The above is how the atrial fibrillation decision process is performed.

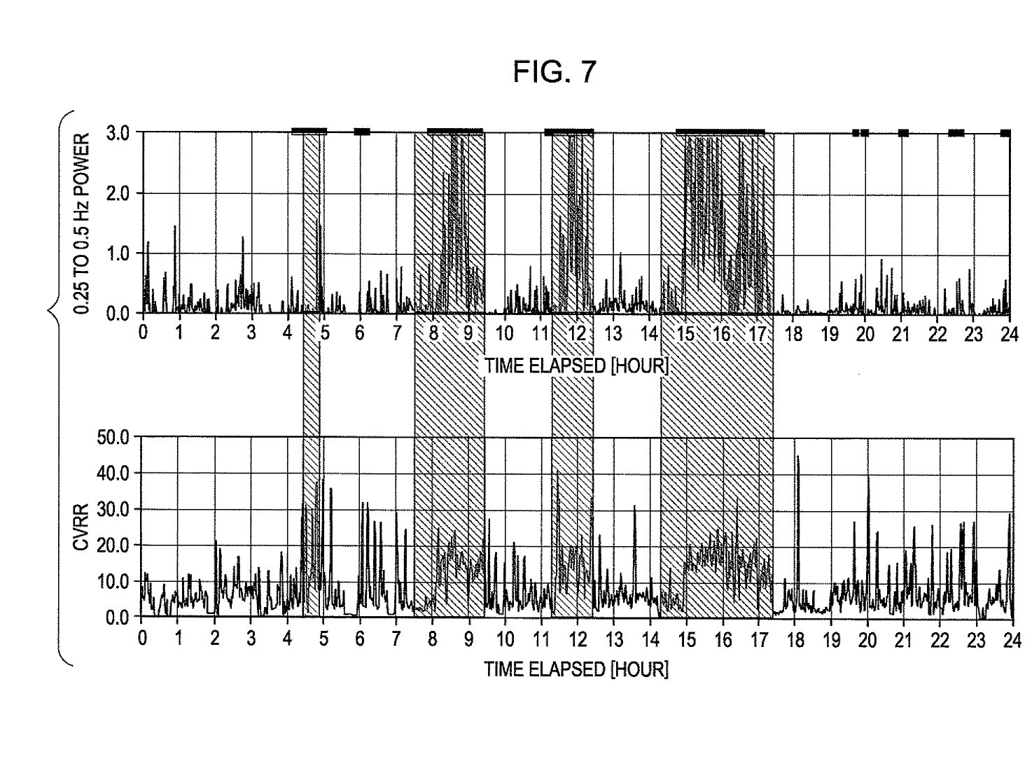

FIG. 7 is a graph showing a decision result according to this embodiment. FIG. 7 shows the analysis result of the signals shown in FIG. 12, as well as the period in which it was decided that atrial fibrillation was taking place through analysis of electrocardiogram based on a Holter electrocardiogram. In FIG. 7, portions expressed by bold lines correspond to the periods in which it was decided that atrial fibrillation was taking place by the method according to this embodiment, and hatched portions correspond to the periods in which it was decided that atrial fibrillation was taking place through analysis of electrocardiogram based on the Holter electrocardiogram. The periods in which it was decided that atrial fibrillation was taking place by the Holter electrocardiogram have also been detected, substantially without exception, by the method according to this embodiment as the periods in which atrial fibrillation was taking place. Although noise is interpreted as atrial fibrillation at a few points in this embodiment, the decision result of atrial fibrillation may be excluded in the case where, for example, the decision is made with respect to a period shorter than a predetermined threshold. In other words, the decision that atrial fibrillation is taking place may be adopted only when the atrial fibrillation continues to be detected for a period longer than the threshold.

Thus, the pulse wave measurement device 1 according to the embodiment of the invention is capable of deciding the presence of atrial fibrillation with reduced impact of body motion noise, by measuring the average RR interval in pulse wave instead of RR intervals in pulse wave with respect to each pulse.

### Variations

Although the embodiment of the invention has been described as above, the invention may be realized in various different manners.

### Variation 1

Although the detected waveform signal L indicates the detection result of the pulse wave provided by the pulse wave detection unit 20 according to the foregoing embodiment, the detected waveform signal L may be a waveform signal obtained through detection of electrocardiogram. In other words, it suffices that the waveform signal allows a parameter corresponding to the RR interval to be acquired.

### Variation 2

Although the atrial fibrillation decision apparatus 100 includes the noise reduction unit 111 according to the foregoing embodiment, this is not mandatory. Instead, the RR interval calculation unit 112 may be configured to acquire the detected waveform signal L for the frequency analysis from the detected waveform signal storage region 121.

### Variation 3

Although the atrial fibrillation decision apparatus 100 is provided in the pulse wave measurement device 1 according to the foregoing embodiment, the atrial fibrillation decision apparatus 100 may be realized in an information processing apparatus such as a personal computer. In this case, the information processing apparatus may acquire the detected waveform signal L measured in advance from an external apparatus, and store the detected waveform signal L in the detected waveform signal storage region 121. Further, the information processing apparatus may analyze the detected waveform signal L through the atrial fibrillation decision process, thus to decide whether atrial fibrillation is taking place.

### Variation 4

Although the main unit 10 and the pulse wave detection unit 20 are connected to each other via the cable 30 according to the foregoing embodiment, those units may be wirelessly connected to each other. In this case, the main unit 10 and the pulse wave detection unit 20 may exchange therebetween the control signals necessary for controlling the pulse wave detection unit 20 and the signals generated by the pulse wave detection unit 20 such as the detected waveform signal L, through wireless communication. In addition, each of the main unit 10 and the pulse wave detection unit 20 may include a battery or the like that can supply power thereto.

### Variation 5

Although the decision result of atrial fibrillation is displayed on the display unit 15 to be notified to the user according to the foregoing embodiment, the notification may be made by a sound or vibration. In the case of employing a sound, for example, a speaker and a sound control unit that controls the sound output of the speaker on the basis of the information from the decision unit 114 may be provided. In the case of employing vibration for notification to the user, a vibration actuator and a vibration control unit that controls the performance of the vibration actuator on the basis of the information from the decision unit 114 may be provided. Thus, the display control unit 115 and the display unit 15 according to the embodiment may be conceived as a notification unit that outputs a notice in accordance with the decision result of atrial fibrillation.

### Variation 6

Various parameters cited in the foregoing embodiment, such as the threshold for clustering (±30%), the predetermined range for the coordinate of average center of gravity (10.0 or higher in fluctuation coefficient and 0.5 or higher in power 0.5), the number of pieces of the data for the +moving average processing (20 pieces), and the length of the frame (120 seconds) are only exemplary and in no way limit the values of those parameters. In addition, the algorithm for the clustering is not limited to the k-means process. The data group may be divided into two clusters by an algorithm other than the k-means process. Further, the specific method of deciding the presence of atrial fibrillation is not limited to the process described with reference to FIG. 11. The decision on the atrial fibrillation may be made by a method other than the process described with reference to FIG. 11, for example through comparison of either of the power or the fluctuation coefficient with the threshold.

### Variation 7

The control program according to the foregoing embodiment may be stored in a computer-readable recording medium such as a magnetic recording medium (magnetic tape, magnetic disk, or the like), an optical recording medium (optical disk or the like), a photomagnetic recording medium, a semiconductor memory, and so forth. Further, the pulse wave measurement device 1 may acquire the programs by downloading through a network.

The entire disclosure of Japanese Patent Application No. 2011-194904, filed Sep. 7, 2011 and 2012-184978, filed Aug. 24, 2012 are expressly incorporated reference herein.

What is claimed is:

1. An atrial fibrillation decision apparatus comprising:
an acquisition unit that acquires a detected waveform signal indicating a detection result of an electrocardiogram or pulse wave;
an RR interval calculation unit that calculates, with respect to each of a plurality of frames, a parameter corresponding to an average RR interval of the frame on the basis of a spectrum of the frame obtained through frequency analysis of the detected waveform signal acquired;
a power calculation unit that calculates a power of a predetermined frequency band in an RR waveform signal indicating a temporal change of the average RR interval calculated by the RR interval calculation unit; and
a decision unit that decides whether the calculated power satisfies a specific condition and outputs information indicating whether atrial fibrillation is taking place, on the basis of the detection result.

2. The atrial fibrillation decision apparatus according to claim 1, further comprising a fluctuation coefficient calculation unit that calculates a fluctuation coefficient of the average RR interval in the RR waveform signal,
wherein the decision unit decides whether a set of the power and the fluctuation coefficient satisfies the specific condition and outputs the information indicating whether atrial fibrillation is taking place in accordance with the decision result.

3. The atrial fibrillation decision apparatus according to claim 2,
wherein the decision unit divides a plurality of sets of the power and the fluctuation coefficient acquired from a plurality of frames into a first cluster of a relatively high power and high fluctuation coefficient and a second cluster of a relatively low power and low fluctuation coefficient, and
decide whether atrial fibrillation is taking place on the basis of a positional relationship between a first center of gravity of the first cluster and a second center of gravity of the second cluster in a fluctuation coefficient-power coordinate.

4. The atrial fibrillation decision apparatus according to claim 3,
wherein the decision unit decides the first cluster as representing a state where atrial fibrillation is taking place, in the case where the first center of gravity and the second center of gravity are spaced from each other by a distance equal to or larger than a first predetermined threshold.

5. The atrial fibrillation decision apparatus according to claim 4,
wherein the decision unit decides the first cluster and the second cluster as representing a state where atrial fibrillation is taking place, in the case where the spacing between the first center of gravity and the second center of gravity is smaller than the first predetermined threshold, and the average center of gravity of the first center of gravity and the second center of gravity has a power equal to or larger than a second predetermined threshold and a fluctuation coefficient equal to or higher than a third predetermined threshold.

6. The atrial fibrillation decision apparatus according to claim 5,
wherein the decision unit decides the first cluster and the second cluster as not representing a state where atrial fibrillation is taking place, in the case where the spacing between the first center of gravity and the second center of gravity is smaller than the first predetermined threshold, and the average center of gravity of the first center of gravity and the second center of gravity does not have either or both of a power equal to or larger than the second predetermined threshold and a fluctuation coefficient equal to or higher than the third predetermined threshold.

7. The atrial fibrillation decision apparatus according to any one of claim 1
wherein a minimum frequency of the frequency band is equal to or higher than the reciprocal of the time of the frame.

8. The atrial fibrillation decision apparatus according to any one of claim 1, further comprising:
a detection unit that detects the electrocardiogram or the pulse wave of the subject; and
a notification unit that notifies the subject of the information outputted by the decision unit,
wherein the acquisition unit acquires the detected waveform signal obtained on the basis of the detection result.

9. The atrial fibrillation decision apparatus according to claim 8,
wherein the acquisition unit includes a noise reduction unit that filters out components originating from body motion noise from the detected waveform signal thus reducing the noise, and outputs the filtered signal as the detected waveform signal.

10. A method of deciding presence of atrial fibrillation, comprising:
acquiring a detected waveform signal indicating a detection result of an electrocardiogram or pulse wave;
calculating, with respect to each of a plurality of frames, a parameter corresponding to an average RR interval of the frame on the basis of a spectrum of the frame obtained through frequency analysis of the detected waveform signal acquired;
calculating a power of a predetermined frequency band in an RR waveform signal indicating a temporal change of the calculated average RR interval;
calculating a fluctuation coefficient of the average RR interval in the RR waveform signal;
deciding whether a set of the power and the fluctuation coefficient satisfies a specific condition; and
outputting information indicating whether atrial fibrillation is taking place, on the basis of the decision result.

11. A program that causes a computer to:
acquire a detected waveform signal indicating a detection result of an electrocardiogram or pulse wave;
calculate, with respect to each of a plurality of frames, a parameter corresponding to an average RR interval of the frame on the basis of a spectrum of the frame obtained through frequency analysis of the detected waveform signal acquired;
calculate a power of a predetermined frequency band in an RR waveform signal indicating a temporal change of the calculated average RR interval;
calculate a fluctuation coefficient of the average RR interval in the RR waveform signal;
decide whether a set of the power and the fluctuation coefficient satisfies a specific condition; and
output information indicating whether atrial fibrillation is taking place, on the basis of the decision result.
